(19) United States
(12) Patent Application Publication
(10) Pub. No.: US 2013/0018194 A1
(43) Pub. Date: Jan. 17, 2013

(54) OCTAHYDROBINAPHTHOL DERIVATIVE FOR L/D OPTICAL CONVERSION AND OPTICAL RESOLUTION

(71) Applicant: Kwan-Mook Kim, Seoul (KR)

(72) Inventor: Kwan-Mook Kim, Seoul (KR)

(73) Assignees: AMINOLUX, INC, Seoul (KR); EWHA UNIVERSITY-INDUSTRY COLLABORATION FOUNDATION, Seoul (KR)

(21) Appl. No.: 13/638,076

(22) PCT Filed: Apr. 1, 2011
(86) PCT No.: PCT/KR11/02301

§ 371 Date: Sep. 28, 2012

(30) Foreign Application Priority Data
Apr. 2, 2010 (KR) 10-2010-0030373

(51) Int. Cl. C07C 227/36; C07C 275/38; C07C 273/18; C07D 233/64; C07C 47/575; C07C 45/64; C07B 57/00; C07C 279/18; C07C 277/08

(52) U.S. Cl. 548/341.5; 564/239; 564/52; 568/441; 568/433; 564/303; 564/304; 562/567; 562/445; 562/573; 562/444; 562/575; 562/561

(57) ABSTRACT

This invention relates to octahydro-binaphthol derivatives, which can recognize amino acids and amino alcohols enantioselectively and transform L-amino acids into D-amino acids and optically resolve amino acids or amino alcohol with high efficiency.

### TECHNICAL FIELD

The present invention relates to octahydro-binaphthol derivatives useful for the optical resolution of amino acids or amino alcohols and for the optical transformation of amino acids from a D-form into an L-form, or vice versa, and to synthetic methods and use thereof.

### BACKGROUND ART

Optically pure amino acids are widely used as ligands of asymmetric catalysts or as starting materials or intermediates necessary for synthesizing various pharmaceuticals and physiologically active substances, and are thus regarded as very important in industrial fields.

The economic method of synthesizing amino acids is known as fermentation. The amino acids are limited to natural L-amino acids. Enantiomerically pure D-amino acids and unnatural amino acids can be synthesized by an enzymatic process and optical resolution, but the prices are 5- to 10-fold higher than those of natural L-amino acids prepared via fermentation and there are difficulties in bulk production. So there have been many trials and efforts for economic bulk production of amino acids.

As part of such efforts, the present inventors have developed a method of forming an imine bond using a binaphthol derivative having an aldehyde group to thereby recognize the chirality of a chiral amino alcohol or amino acid and transform L-amino acids into D-amino acids as described below ((a) Park, H.; Kim, K. M.; Lee, A.; Ham, S.; Nam, W.; Chin, J. J. Am. Chem. SC. 2007, 129, 1518-1519; (b) Kim, K. M.; Park, H.; Kim, H.; Chin, J.; Nam, W. Org. Lett. 2005, 7, 3525-3527.).

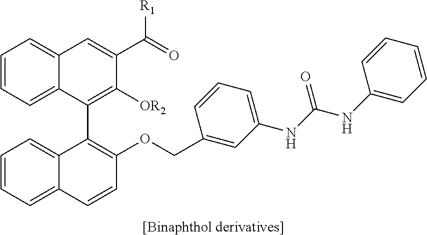

The binaphthol derivative described above was invented based on the reaction mechanism of a PLP compound acting as a cofactor in an enzyme called amino acid racemase. The method of obtaining pure optical isomers of amino acids or amino alcohols using these binaphthol derivatives can be applied broadly and economically comparing with the conventional enzymatic process or classical optical resolution, and is expected to be a good alternative to obtain novel amino acid and amino alcohol optical isomers.

### DISCLOSURE

### Technical Problem

Accordingly, the present invention provides octahydro-binaphthol derivatives for optical (L/D) conversion of amino acids and optical resolution of amino acids or amino alcohols, which show good solubility and higher chiral selectivity due to the restriction of dihedral angle between rings compared to conventional binaphthol derivatives, and their synthetic methods. In addition, the present invention provides the methods of L/D optical conversion of amino acids and optical resolution of amino alcohols.

### Technical Solution

The present invention provides octahydro-binaphthol derivatives represented by Formula 1 below.

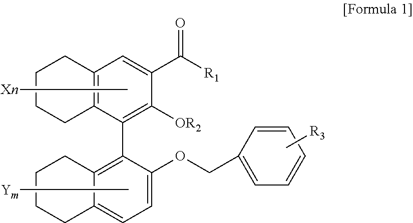

In Formula 1, X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

n and m are an integer from 0 to 5, respectively;

R1 and R2 are independently C1˜C5 alkyl unsubstituted or substituted with halogen or hydroxyl; C3˜C10 cyclic alkyl unsubstituted or substituted with halogen or hydroxyl; C2˜C5 alkenyl unsubstituted or substituted with halogen or hydroxyl; C2˜C5 alkynyl unsubstituted or substituted with halogen or hydroxyl; or aryl unsubstituted or substituted with halogen or hydroxyl;

R3 is —NHCZR4, —NHS(O)ₐR4, —NHPO(OH)R4 or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1˜C5 perfluoroalkyl, R6 and R7 can form a cycle, and when R3 is —NHC(NH₂)NH₂⁺ or —NHCHNH₂⁺, a counter anion is a halogen ion or R10COO⁻ and R10 is C1˜C10 alkyl or C5˜C12 aryl unsubstituted or substituted with C1˜C5 alkyl; and

alkyls described above are linear or branched alkyls.

Also the present invention provides a method of synthesizing the octa-hydro-binaphthol derivatives of Formula 1.

Also the present invention provides an intermediate compound represented by Formula 4 below used to prepare the octahydro-binaphthol derivatives of Formula 1.

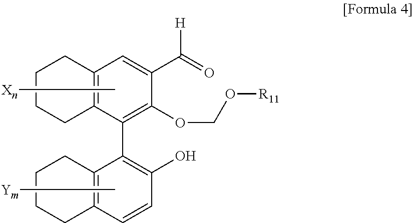

In Formula 4, X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

n and m are an integer from 0 to 5, respectively;

R11 is C1˜C5 alkyl, C6˜C10 arylalkyl or C2˜C10 alkoxyalkyl; and

alkyls described above are linear or branched alkyls.

Also the present invention provides a method of optical resolution of racemic amino alcohols or racemic amino acids using the octahydro-binaphthol derivatives of Formula 1.

Also the present invention provides a method of optical conversion from a D-form to an L-form or vice versa using the octahydro-binaphthol derivatives of Formula 1.

### Advantageous Effects

The present invention using novel octahydro-binaphthol derivatives shows higher chiral selectivity due to the restriction of dihedral angle between the rings compared to conventional binaphthol derivatives, and can be useful for optical (L/D) conversion of amino acids and optical resolution of amino acids or amino alcohols. In addition, good solubility of octahydro-binaphthol derivatives is very convenient for use.

### DESCRIPTION OF DRAWINGS

FIG. 1 shows energy-minimized structures of corresponding imines formed by the reaction of an octahydro-binaphthol derivative (compound 2) or a conventional binaphthol derivative with (R)-2-aminopropanol, respectively and shows dihedral angles between two phenyl rings of the corresponding imines;

FIG. 2 shows ¹H NMR spectrums which show the chiral selectivity for amino alcohols using an octahydro-binaphthol derivative (compound 3) of the present invention [(a): compound 3, (b): compound 3-S-ap, (c): compound 3-R-ap, (d): compound 3 and racemic (RS)-ap]; and

FIG. 3 shows ¹H NMR spectrums which show the optical (L/D) conversion of an amino acid by the octahydro-binaphthol derivative (compound 2) of the present invention.

### BEST MODE

The present invention pertains to octahydro-binaphthol derivatives represented by Formula 1 below.

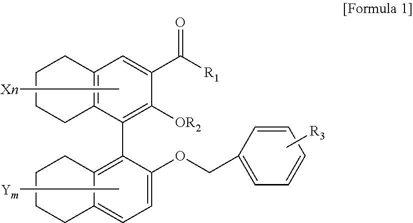

In Formula 1, X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

n and m are an integer from 0 to 5, respectively;

R1 and R2 are independently C1˜C5 alkyl unsubstituted or substituted with halogen or hydroxyl; C3˜C10 cyclic alkyl unsubstituted or substituted with halogen or hydroxyl; C2˜C5 alkenyl unsubstituted or substituted with halogen or hydroxyl; C2˜C5 alkynyl unsubstituted or substituted with halogen or hydroxyl; or aryl unsubstituted or substituted with halogen or hydroxyl;

R3 is —NHCZR4, —NHS(═O)ₐR4, —NHPO(OH)R4 or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1˜C5 perfluoroalkyl, R6 and R7 can form a cycle, and when R3 is —NHC(NH₂)NH₂⁺or —NHCHNH₂⁺, a counter anion is a halogen ion or R10COO⁻ and R10 is C1˜C10 alkyl or C5˜C12 aryl unsubstituted or substituted with C1˜C5 alkyl; and

alkyls described above are linear or branched alkyls.

In Formula 1, R3 is preferably —NHC(═O)NH₂—C₆H₅, or —NHC(NH₂)NH₂⁺.

Representative examples of the compound of Formula 1 are as follows.

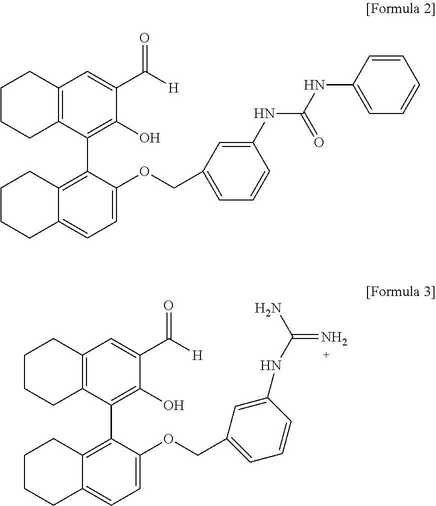

Typically, octahydro-binaphthol derivatives are advantageous because the dihedral angle between rings is limited to thus show better chiral selectivity compared to binaphthol derivatives, and also because of good solubility.

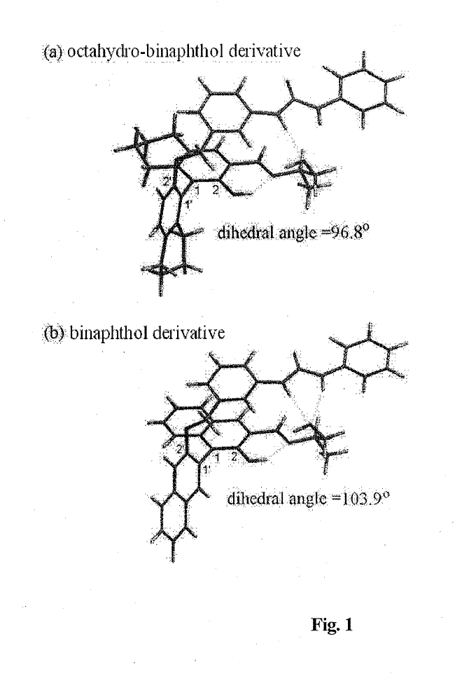

Amino alcohol and the compound of Formula 1 can make imines. In FIG. 1, the compound of Formula 2 that is the octahydro-binaphthol derivative and the binaphthol derivative (2-hydroxy-2′-(3-phenyluryl-benzyloxy)-3-formyl-1,1′-binaphthalene) respectively form imines along with (R)-2-amino-1-propanol, which are represented by the energy-minimized structures obtained through Molecular Mechanics calculations using the SPARTAN program. In the dihedral angle between the two phenyl rings of each imine, the binaphthol derivative by 103.9° and the octahydro-binaphthol derivative by 96.8° are different. As a result, the selectivity for chiral amino alcohols is affected. Moreover, typically octahydro-binaphthol derivatives have higher solubility in organic solvents compared to binaphthol derivatives and are thus advantageous in terms of applications and functions.

In the present invention, the compound of Formula 1 is used in an optically pure form.

The optically pure compound of Formula 1 may be used as an (R)-form as well as an (S)-form.

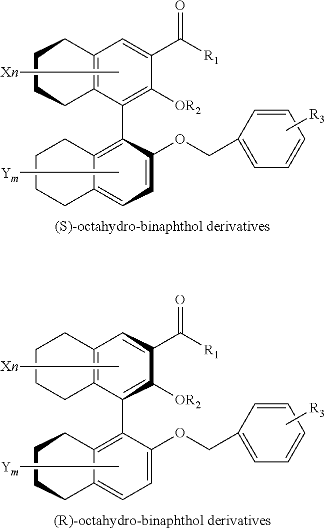

Also the present invention relates to a method of preparing the compound of Formula 1, which includes reacting compounds represented by the following Formula 4 and Formula 5 in the presence of a base.

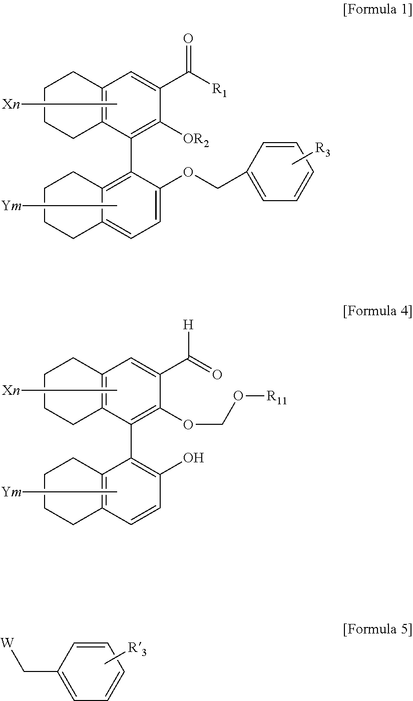

In the above formulas, X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

n and m are an integer from 0 to 5, respectively;

R1 and R2 are independently C1˜C5 alkyl unsubstituted or substituted with halogen or hydroxyl; C3˜C10 cyclic alkyl unsubstituted or substituted with halogen or hydroxyl; C2˜C5 alkenyl unsubstituted or substituted with halogen or hydroxyl; C2˜C5 alkynyl unsubstituted or substituted with halogen or hydroxyl; or aryl unsubstituted or substituted with halogen or hydroxyl;

R3 is —NHCZR4, —NHS(═O)ₐR4, —NHPO(OH)R4 or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1˜C5 perfluoroalkyl, R6 and R7 can form a cycle, and when R3 is —NHC(NH₂)NH₂⁺or —NHCHNH₂⁺, a counter anion is a halogen ion or R10COO⁻ and R10 is C1˜C10 alkyl or C5˜C12 aryl unsubstituted or substituted with C1˜C5 alkyl;

R3′ is NO₂, —NHCZR4, —NHS(═O)ₐR4, —NHPO(OEt)R4, —NHC(NHBOC)(NBOC) or —NHC(NHR6)⁺R5, wherein Z is oxygen or sulfur, a is 1 or 2, and R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1˜C5 perfluoroalkyl, R6 and R7 can form a cycle, and when R3 is —NHC(NH₂)NH₂⁺or —NHCHNH₂⁺, a counter anion is a halogen ion or R10COO⁻ and R10 is C1˜C10 alkyl or C5˜C12 aryl unsubstituted or substituted with C1˜C5 alkyl;

R11 is C1˜C5 alkyl, C6˜C10 arylalkyl or C2˜C10 alkoxyalkyl;

W is halogen; and

alkyls described above are linear or branched alkyls.

Also the present invention relates to an intermediate compound used in the preparation of the compound of Formula 1, which is represented by Formula 4 below.

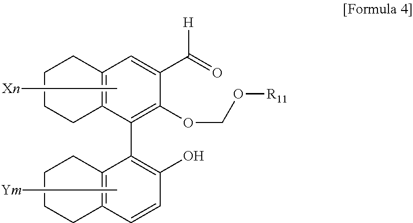

In Formula 4,

X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 to alkylcarbonyl; C6˜C10 aryl; and C1˜C10 alkoxy;

n and m are an integer from 0 to 5, respectively;

R11 is C1˜C5 alkyl, C6˜C10 arylalkyl or C2˜C10 alkoxyalkyl; and

alkyls described above are linear or branched alkyls.

Also the present invention relates to a method of preparing the compound of Formula 4, which includes reacting compounds represented by the following Formula 6 and Formula 7 in the presence of a base.

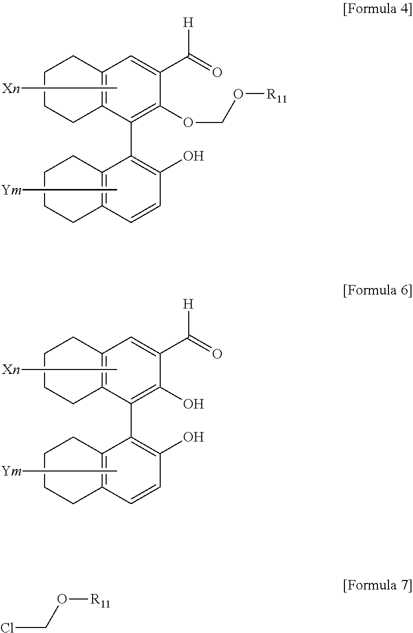

In the above formulas, X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;

Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1- C10 alkoxy;

n and m are an integer from 0 to 5, respectively;

R11 is C1˜C5 alkyl, C6˜C10 arylalkyl or C2˜C10 alkoxyalkyl; and

alkyls described above are linear or branched alkyls.

The compound of Formula 6 can be prepared by the reaction of paraformaldehyde with a compound of Formula 8 below.

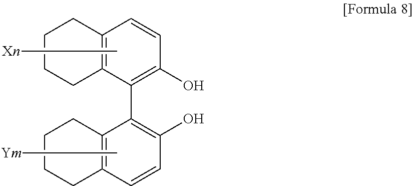

The method of preparing the octahydro-binaphthol derivatives of the invention will be described in further detail using the method of preparing the optical (S)-form compound 2 and compound 3.

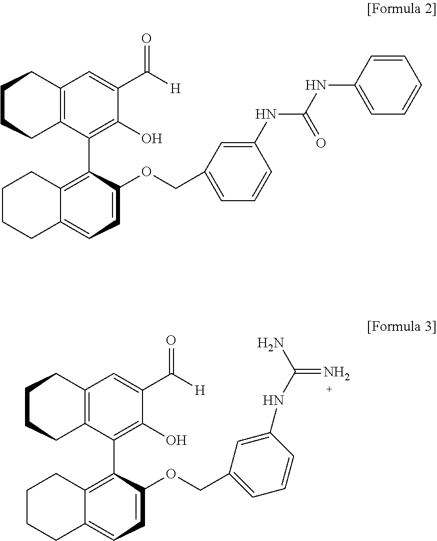

Any method of synthesizing the compound 2 can be applied, but this compound can be typically synthesized via the following Scheme 1.

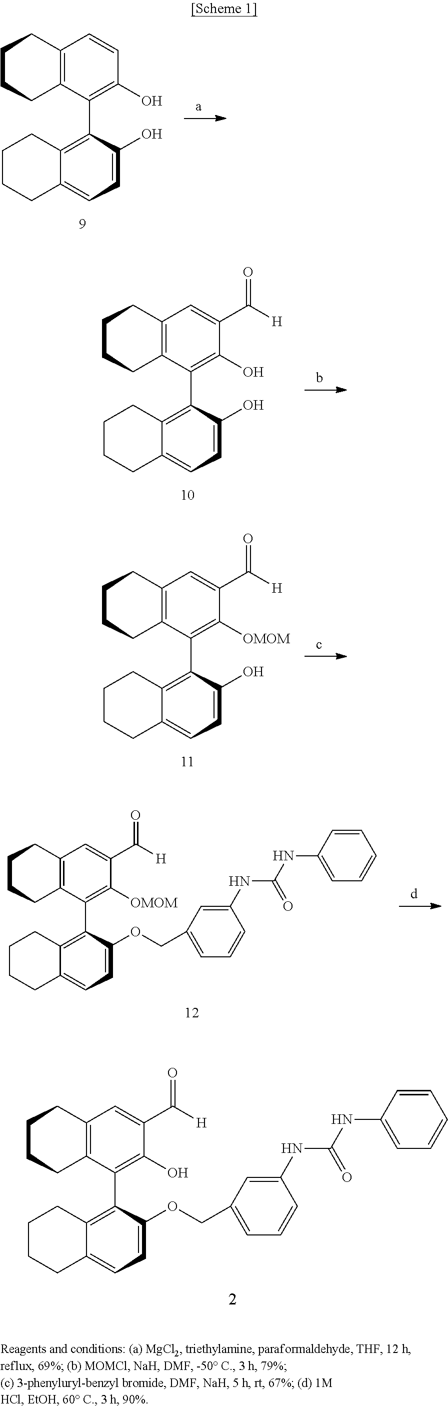

The commercially available octahydro-binaphthol (compound 9) is mixed with MgCl₂, triethylamine and paraformaldehyde, and boiled for 12 hours, and thereby compound 10 in which the aldehyde group is introduced next to the —OH group of the phenyl ring is made. When the compound 10 is reacted with one equivalent of each of NaH and methoxy methyl (MOM) chloride, the MOM is attached to —OH next to the aldehyde group (compound 11). The reaction of the compound 11 with NaH and 3-phenyluryl-benzyl bromide results in compound 12, after which MOM is added dropwise under an acidic condition, thereby obtaining compound 2.

Any method of preparing the compound of Formula 3 can be applied, but this compound can be synthesized via Scheme 2 below.

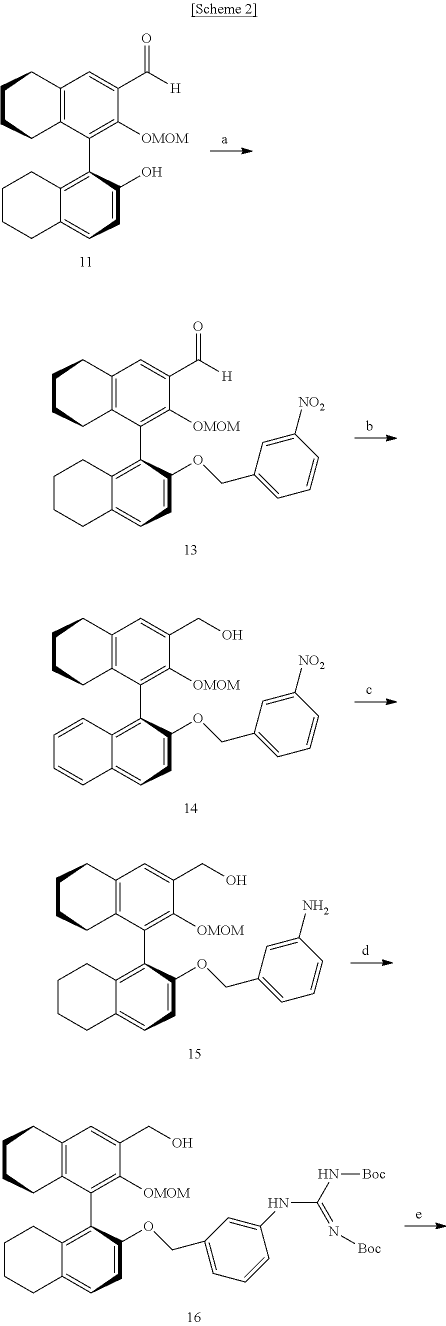

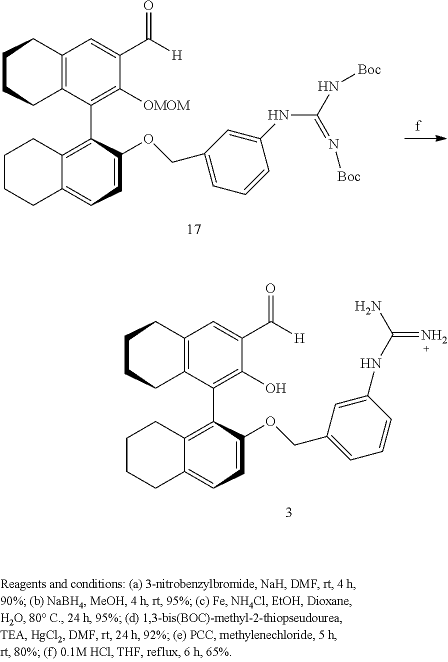

When compound 11 is reacted with 3-nitrobenzyl bromide and NaH, compound 13 is obtained, followed by reduction of the aldehyde group by NaBH₄ and nitro reduction by Fe and NH₄Cl, thus continuously obtaining compounds 14 and 15. Compound 15 is reacted with 1,3-bis(BOC)-2-methyl-2-thiopseudourea, thus obtaining compound 16. Finally, alcohol is oxidized to aldehyde by PCC, thus obtaining compound 17, followed by acid treatment, resulting in compound 3.

Also the present invention relates to a method of optical resolution of racemic amino alcohols or racemic amino acids using the compound of Formula 1.

Also the present invention provides a method of optical conversion from a D-amino acid to an L-amino acid or vice versa using the compound of Formula 1.

The octahydro-binaphthol derivatives of the present invention have functional groups that can form the imine via reaction with various amines, and the principle of optical resolution depends on differences in the stability of the imine compounds.

Examples of the amino alcohols which may be optically resolved by the compound of the invention include, but are not limited to, b- or g-amino alcohols having a monovalent amine group. A typical example of the b- or g-amino alcohols having a monovalent amine group includes a compound of Formula 18 below, which includes optical isomers of R-form or S-form because of chiral carbon in the molecule.

H₂NCHR₁₂CR₁₃R₁₄OH [Formula 18]

wherein R12 is independently a monovalent organic group or halogen, excluding hydrogen, and is preferably substituted or unsubstituted alkyl, substituted or unsubstituted alkenyl, substituted or unsubstituted cyclic alkyl, or substituted or unsubstituted aryl, and R13 and R14 are independently hydrogen, substituted or unsubstituted alkyl, substituted or unsubstituted alkenyl, substituted or unsubstituted cyclic alkyl, or substituted or unsubstituted aryl.

The octahydro-binaphthol derivative of the invention enables optical resolution of α-amino acid and β-amino acid. Examples of the amino acid may include, but are not limited to, a- or b-amino acids. Typical examples of the a- or b-amino acids include amino acids represented by Formula 19 or 20 below.

H₂NCHR₁₅COOH [Formula 19]

H₂NCHR₁₅CHR₁₆COOH [Formula 20]

wherein R15 and R16 are each independently a monovalent organic group or halogen, excluding hydrogen, and preferably substituted or unsubstituted alkyl, substituted or unsubstituted alkenyl, substituted or unsubstituted cyclic alkyl, or substituted or unsubstituted aryl.

As the method for optical resolution of racemic amino alcohols or racemic amino acids using the octahydro-binaphthol derivatives of the invention, any method may be used as long as it is known in the art. That is, a batch process using a solvent, a column process in which a column is filled with the compound, etc., may be applied. Further, the amino alcohol or amino to acid, which is subjected to primary optical resolution, may be repeatedly subjected to optical resolution, if required, thereby obtaining amino alcohols or amino acids having higher optical purity.

In the case of the (S)-form optical isomer of the compound of Formula 1 of the invention, an L-amino acid may be transformed into a D-amino acid. In the case of the (R)-form optical isomer, a D-amino acid may be transformed into an L-amino acid. This phenomenon is considered to be due to the recognition of chirality of the chiral compound.

### MODE FOR INVENTION

A better understanding of the present invention may be obtained through the following examples, which are set to illustrate, but are not to be construed as limiting the present invention. The following examples can be appropriately modified and changed by those skilled in the art within the scope of the present invention.

### Example 1

### Synthesis of (S)-2,2′-hydroxy-3-formyl-5,5′,6,6′,7,7′,8,8′-octahydro-1,1′-binaphthalene (compound 10)

To anhydrous THF (300 ml), (S)-5,5′,6,6′,7,7′,8,8′-octahydro-1,1′-binaphthol (compound 9, 12 g, 40.76 mmol), anhydrous MgCl₂ (7.76 g, 81.52 mmol), triethylamine (11.36 ml, 81.52 mmol) and paraformaldehyde (4 g) were added and refluxed for 12 hours. The reaction was monitored by TLC. After completion of the reaction, aq. HCl was added. The reaction solution was evaporated and extracted with ethyl acetate/water. The organic layer was dried over MgSO₄ and concentrated, followed by column chromatography on silica gel (ethyl acetate:hexane=1:1) to give the pure compound 10.

Yield: 8.5 g (69%); mp 146-147□; ¹H NMR (CDCl₃, 250 MHz) δ (ppm)=10.89 (s, 1H, —CHO), 9.86 (s, 1H, —OH), 7.35-6.73 (s, dd, 3H), 4.37 (s, 1H, —OH), 2.82-1.66 (m, 16H); ¹³C NMR (CDCl₃, 63 MHz) δ (ppm)=192.3, 165.4, 157.3, 155.7, 148.6, 137.9, 136.3, 132.5, 128.5, 123.3, 120.3, 28.7, 27.9, 27.2, 27.0, 23.9, 23.5, 23.0, 22.9.

### Example 2

### Synthesis of (S)-2-methoxyethoxy-2′-hydroxy-3-formyl-5,5′,6,6′,7,7′,8,8′-octahydro-1,1′-binaphthalene (compound 11)

The mixture of compound 10 (6.5 g, 21 mmol) of Example 1 and 60% NaH (0.79 g, 20 mmol) in DMF (50 ml) was stirred for 1 h. Chloromethyl methyl ether (MOMCl) (2.36 g, 31 mmol) was added and stirred for 3 h at room temperature. After the reaction was completed, the reaction mixture was extracted with ethyl acetate/water. The organic layer was dried over MgSO₄ and concentrated, followed by recrystallization from the mixture of ethyl acetate and hexane to give compound 11.

Yield: 6.0 g (79%); mp 139-140□; ¹H NMR (CDCl₃, 250 MHz): δ (ppm)=10.26 (s, 1H, —CHO), 7.66-6.76 (s, dd, 3H), 4.81-4.69 (dd, 2H, —OCH₂), 4.54 (s, 1H, —OH), 3.13 (s, 3H, —OCH₃), 2.86-1.65 (m, 16H); ¹³C NMR (CDCl₃, 63 MHz): δ (ppm)=190.9, 156.9, 155.9, 150.7, 148.0, 146.4, 136.1, 134.6, 130.0, 129.9, 127.3, 122.3, 100.2, 57.2, 29.5, 27.9, 27.8, 27.4, 27.1, 23.2, 23.0, 22.6, 22.4.

### Example 3

### Synthesis of (S)-2-methoxyethoxy-2′-(3-phenyluryl-benzyloxy)-3-formyl-5,5′,6,6′,7,7′,8,8′-octahydro-1,1′-binaphthalene (compound 12)

The mixture of compound 11 (1 g, 2.73 mmol) of Example 2 and NaH (0.1 g, 2.45 mmol) in DMF (5 ml) was stirred for 30 min. 3-phenylurylbenzyl bromide (0.58 g, 2.73 mmol) was added. After stirring for 1 h, the reaction mixture was concentrated and extracted with ethyl acetate, followed by column chromatography on silica gel (ethyl acetate:hexane=1:5) to give compound 12.

Yield: 1.01 g (67%); mp 195-196□; ¹H NMR (CDCl₃, 250 MHz) δ (ppm)=10.27 (s, 1H, CHO), 7.57-6.66 (m, 12H), 4.97-4.95 (d, 2H, OCH₂), 4.72-4.71 (dd, 2H, OCH₂O), 2.96 (s, 3H, OCH₃), 2.81-1.59 (m, 16H); ¹³C NMR (CDCl₃, 63 MHz) δ (ppm)=189.7, 162.1, 155.6, 154.7, 149.0, 139.1, 138.4, 137.5, 136.1, 135.4, 130.8, 129.9, 129.3, 128.4, 125.1, 123.7, 123.2, 121.3, 120.7, 119.3, 118.4, 118.0, 117.5, 72.4, 67.5, 31.3, 29.3, 29.8, 29.1, 28.6, 23.5, 23.2, 22.9, 22.6.

### Example 4

### Synthesis of (S)-2-hydroxy-2′-(3-phenyl-benzyloxy)-3-formyl-5,5′,6,6′,7,7′,8,8′-octahydro-1,1′-binaphthalene (compound 2)

Compound 12 (1 g, 1.829 mmol) of Example 3 was dissolved in ethanol. HCl (0.48 ml, 5.49 mmol) was added and refluxed for 3 h. The reaction mixture was concentrated, followed by column chromatography on silica gel (ethyl acetate:hexane=1:5) to give compound 2.

Yield: 0.89 g (90%); mp 117-118□; ¹H NMR (CDCl₃, 250 MHz): δ (ppm)=10.73 (s, 1H, CHO), 9.69 (s, 1H, OH), 7.43-6.70 (m, 12H), 4.88 (s, 2H, OCH₂), 2.75-1.65 (m, 16H); ¹³C NMR (CDCl₃, 63 MHz): δ (ppm)=196.4, 156.1, 153.7, 153.3, 147.3, 138.5, 138.2, 138.1, 136.8, 133.2, 130.4, 129.7, 129.4, 128.9, 125.6, 123.9, 123.4, 121.8, 120.3, 119.7, 118.9, 118.7, 110.7, 70.0, 29.3, 29.0, 28.1, 27.0, 23.0, 22.9, 22.7, 22.5; HRMS (FAB) calcd for C₃₅H₃₆N₂O₄: 548. 2675. found: 548.2669.

### Example 5

### Synthesis of (S)-2-methoxyethoxy-2′-(3-nitrobenzyloxy)-3-formyl-5,5′,6,6′,7,7′,8,8′-octahydro-1,1′-binaphthalene (compound 13)

The mixture of compound 11 (3.0 g, 8.19 mmol) and 60% NaH (0.33 g, 8.19 mmol) in DMF (50 ml) was stirred for 30 min. 3-nitrobenzylbromide (2.12 g, 8.19 mmol) was added and the mixture was stirred for 4 h. After the reaction was completed, the reaction mixture was extracted with ethyl acetate, dried over MgSO₄ and concentrated, followed by column chromatography on silica gel (ethyl acetate:hexane=1:5) to give compound 13.

Yield: 4.0 g (90%); mp 110-112□; ¹H NMR (CDCl₃, 250 MHz): δ (ppm)=10.29 (s, 1H, —CHO), 8.10-6.74 (m, 7H), 5.09 (s, 2H, —OCH₂), 4.77-4.62 (dd, 2H, —OCH₂O), 3.08 (s, 3H, —OCH₃), 2.88-1.68 (m, 16H); ¹³C NMR (CDCl₃, 63 MHz): δ (ppm)=191.8, 163.4, 153.3, 141.8, 137.3, 136.7, 133.9, 132.5, 131.3, 129.2, 129.0, 128.8, 128.2, 127.5, 126.1, 122.4, 120.8, 109.3, 98.5, 72.5, 68.2, 31.5, 29.6, 28.7, 28.1, 27.3, 26.7, 22.8, 22.4, 21.7, 21.3.

### Example 6

### Synthesis of (S)-2-methoxyethoxy-2′-(3-nitrobenzyloxy)-3-hydroxymethyl-5,5′,6,6′,7,7′,8,8′-octahydro-1,1′-binaphthalene (compound 14)

The mixture of compound 13 (4.0 g, 8.0 mmol) of Example 5 and sodium borohydride (0.36 g, 9.57 mmol) in methanol was stirred for 4 h. After the reaction was completed, the reaction mixture was concentrated and extracted with ethyl acetate to give compound 14.

Yield: 3.8 g (95%); mp 138-139□; ¹H NMR (CDCl₃, 250 MHz): δ (ppm)=8.09-6.73 (m, 7H), 5.09 (s, 2H, —OCH₂), 4.73-4.5 (m, 2H, CH₂OH), 4.69-4.49 (dd, 2H, —OCH₂O), 3.26 (s, 3H, —OCH₃), 3.1 (t, 1H, —OH), 2.82-1.63 (m, 16H); ¹³C NMR (CDCl₃, 63 MHz): δ (ppm)=151.8, 150.4, 147.3, 138.8, 136.3, 135.7, 132.9, 131.2, 130.7, 129.8, 129.6, 129.1, 128.3, 128.0, 125.1, 121.4, 120.2, 108.4, 97.9, 67.5, 60.5, 55.8, 30.9, 28.6, 28.4, 28.3, 26.3, 26.0, 22.0, 21.9, 21.6, 21.2.

### Example 7

### Synthesis of (S)-2-methoxyethoxy-2′-(3-naminobenzyloxy)-3-hydroxymethyl-5,5′,6,6′,7,7′,8,8′-octahydro-1,1′-binaphthalene (compound 15)

Compound 14 (4.5 g, 8.84 mmol) of Example 6, iron powder (3.5 g, 62.6 mmol) and ammonium chloride (0.86 g, 16 mmol) were added to a solvent mixture of ethanol, 1,4-dioxane and water (1:1:1), and the mixture was stirred for 24 h. The reaction mixture was concentrated and chromatographed on silica gel (ethyl acetate:hexane=1:5) to give compound 15.

Yield: 4.23 g (95%); mp 132-133□; ¹H NMR (CDCl₃, 250 MHz): δ (ppm)=7.10-6.35 (m, 7H), 4.80-4.70 (m, 2H, CH₂OH), 4.49-4.44 (dd, 2H, —OCH2O), 4.92 (s, 2H, —OCH2), 3.28 (s, 3H, —OCH₃), 2.79-1.61 (m, 16H); ¹³C NMR (CDCl₃, 63 MHz): δ (ppm)=163.8, 163.5, 153.5, 151.4, 138.7, 138.5, 137.0, 136.9, 136.6, 133.6, 131.6, 130.9, 130.1, 129.9, 129.0, 125.8, 122.9, 121.8, 120.1, 119.9, 109.6, 99.1, 98.9, 69.4, 61.5, 56.8, 29.7, 29.5, 29.4, 28.1, 27.3, 27.2, 23.1, 23.0, 22.9.

### Example 8

### Synthesis of (S)-2-methoxyethoxy-3-hydroxymethyl-2′-(3-(N,N-di-tert-butoxycarbonyl-guanidino)benzyloxy)-5,5′,6,6′,7,7′,8,8′-octahydro-1,1′-binaphthalene (compound 16)

Compound 15 (2.5 g, 5.31 mmol) of Example 7 and 1,3-bis-BOC-2-methyl-2-thiopseudourea (1.57 g, 5.31 mmol) were dissolved in DMF. Triethylamine (3.0 ml, 21.24 mmol) and HgCl₂ (1.58 g, 5.84 mmol) were added. The reaction mixture was stirred for 3 h, extracted with ethyl acetate, concentrated and chromatographed on silica gel (ethyl acetate:hexane=1:3) to give compound 16.

Yield: 3.5 g (92%); mp 174-175□; ¹H NMR (CDCl₃, 250 MHz): δ (ppm)=11.65 (s, 1H, NH), 10.21 (s, 1H, NH), 7.62-6.73 (m, 7H), 4.99 (s, 2H, OCH₂), 4.71-4.69 (m, 2H, CH₂OH), 4.48-4.46 (dd, 2H, OCH₂O), 3.25 (s, 3H, OCH₃), 2.79-1.62 (m, 16H), 1.55-1.49 (m, 18H); ¹³C NMR (CDCl₃, 63 MHz): δ (ppm)=163.6, 155.1, 153.5, 152.9, 151.3, 145.2, 137.8, 136.6, 135.4, 133.7, 133.1, 130.5, 129.7, 129.1, 127.3, 126.7, 123.7, 122.2, 120.4, 119.8, 109.4, 99.4, 83.7, 69.5, 54.9, 28.3, 28.1, 27.8, 27.0, 26.6, 26.1, 22.8, 21.5, 21.2.

### Example 9

### Synthesis of (S)-2-methoxyethoxy-2′-(3-(N,N′-di-tertbutoxycarbonyl-guanidino)benzyl)-3-formyl-5,5′,6,6′,7,7′,8,8′-octahydro-1,1′-binaphthalene (compound 17)

The mixture of compound 16 (3.5 g, 4.89 mmol) of Example 8 and PCC (2.1 g, 9.78 mmol) in methylene chloride was stirred for 12 h. The reaction mixture was filtered over celite and the filtrate was concentrated, followed by column chromatography on silica gel (ethyl acetate:hexane=1:5) to give compound 17.

Yield: 2.7 g (80%); mp 86-88□; ¹H NMR (CDCl₃, 250 MHz): δ (ppm)=11.65 (s, 1H, NH), 10.36 (s, 1H, CHO), 10.24 (s, 1H, NH), 7.63-6.75 (m, 7H), 5.00 (s, 2H, OCH₂), 4.78-4.61 (dd, 2H, OCH₂O), 3.09 (s, 3H, OCH₃), 2.85-1.64 (m, 16H), 1.55-1.49 (m, 18H); ¹³C NMR (CDCl₃, 63 MHz): δ (ppm)=189.9, 162.5, 154.3, 152.5, 152.2, 144.3, 137.2, 136.0, 135.8, 133.1, 133.0, 130.8, 129.1, 128.3, 126.9, 126.4, 123.6, 121.7, 120.7, 118.9, 108.5, 98.9, 82.7, 78.6, 68.3, 56.1, 28.6, 28.4, 28.3, 27.0, 26.9, 26.3, 22.0, 21.8, 21.6.

### Example 10

### Synthesis of (S)-2-hydroxy-2′-(3-guanidylbenzyloxy)-3-formyl-5,5′,6,6′,7,7′,8,8′-octahydro-1,1′-binaphthalene (compound 3)

The mixture of compound 17 (0.3 g, 0.42 mmol) of Example 9 and 0.1M conc-HCl (0.84 ml) in THF (5 ml) was stirred for 6 h at 70□. The reaction mixture was concentrated and extracted with ethyl acetate, to give compound 3.

Yield: 0.15 g (65%); mp 159-160□; ¹H NMR (CDCl₃, 250 MHz): 8(ppm)=10.83 (s, 1H, CHO), 9.87 (s, 1H, OH), 9.75 (s, 1H, NH), 7.42-6.77 (m, 7H), 4.94 (s, 2H, OCH₂), 2.86-1.67 (m, 16H); ¹³C NMR (CDCl₃, 63 MHz): δ (ppm)=196.7, 156.6, 156.1, 152.9, 147.0, 140.0, 136.9, 133.9, 133.4, 130.8, 130.1, 129.6, 129.3, 125.9, 125.3, 124.6, 124.5, 123.5, 118.6, 111.1, 69.5, 29.3, 29.0, 28.1, 27.0, 23.0, 22.9, 22.7, 22.6, 21.0; HRMS (FAB) calcd for C₂₉H₃₄N₃O₃: 472.2595. found: 472.2588.

### Test Example 1

### Determination of Chiral Selectivity for Amino Alcohol Using Compound 2 or 3

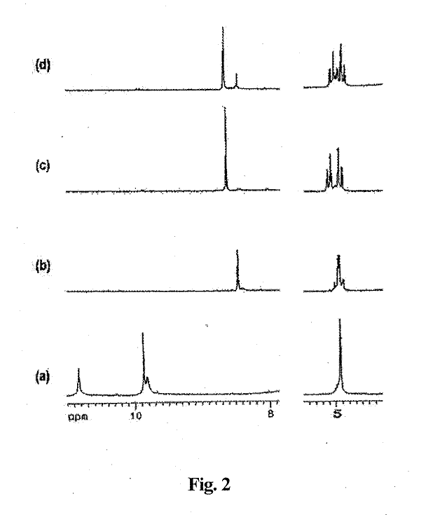

The octahydro-binaphthol derivative (compound 2 or 3) and amino alcohol form an imine. When the equilibrium constant of imine formation with R-amino alcohol is K_{R} and that of imine formation with S-amino alcohol is K_{S}, K_{R}/K_{S} is chiral selectivity, which is determined by ¹H NMR analysis. FIG. 2(a) shows ¹H NMR spectrum of compound 3 near imine CH (s, 9.90 ppm) and benzylic —CH₂— (dd, 4.92).

The two broad peaks at 10.85 and 9.83 ppm are regarded as signals of —OH adjacent to aldehyde and NH of guanidinium. FIG. 2(b) shows ¹H NMR spectrum of compound 3-S-ap formed by the reaction of compound 3 with (S)-2-amino-1-propanol (S-ap) and the singlet peak at 8.5 ppm is the signal by imine hydrogen, which showed that the aldehyde was completely transformed to imine. The benzylic peaks (—CH₂—) at 4.95 ppm showed a doublet of a doublet pattern. FIG. 2(c) shows the spectrum of the imine (3-R-ap) formed by the reaction of compound 3 and (R)-2-amino-1-propanol (R-sp) and shows distinct doublets of doublet patterns of a benzylic (—CH₂—) peak; FIG. 2(d) shows the spectrum obtained from the reaction of compound 3 and 2 equivalents of racemic (RS)-ap for about 1 h. Compound 3-R-ap was produced 4 times more than the 3-S-ap, and based on this, K_{R}/K_{S}=4²=16.0. For other amino acids, chiral selectivities were measured by the same method. The results are given in Table 1 below.

TABLE 1

K_{R}/K_{S}

| Amino alcohol | 2 | 3 |
|---|---|---|
| 2-amino-1-propanol | 3.8 | 16.0 |
| 2-amino-1-butanol | 4.5 | 13.2 |
| Phenylalaninol | 3.7 | 9.8 |
| Valinol | 3.1 | 19.4 |
| Leucinol | 1.8 | 14.4 |

### Test Example 2

### Chiral Transformation of Amino Acids Using Compound 2 or 3

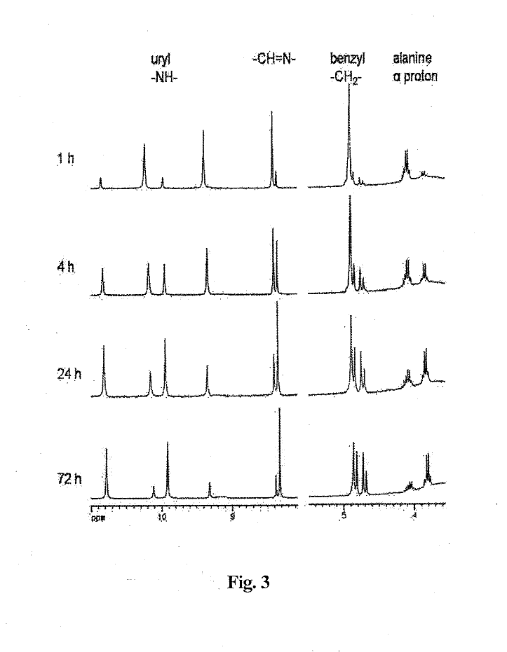

FIG. 3 shows that the imine (2-L-Ala) formed by the reaction of compound 2 and L-alanine is gradually converted to 2-D-Ala during the reaction time. In FIG. 3, the reaction solution reached equilibrium after 72 h and then the ratio of 2-D-Ala/2-L-Ala was 4.0. That is, D-alanine was produced 4 times more than L-Ala. For other amino acids, D/L ratios were measured by the same method. The results are given in Table 2 below.

TABLE 2

D/L ratio

| Amino acids | Compound 2 | Compound 3 |
|---|---|---|
| Ala | 4.0 | 4.4 |
| Asp | 8.0 | 10.1 |
| Glu | 6.1 | 8.0 |
| His | 8.7 | 4.3 |
| Phe | 6.5 | 4.5 |
| Ser | 7.9 | 5.6 |
| Tyr | 8.4 | 4.9 |

1. An octahydro-binaphthol derivative represented by Formula 1 below: 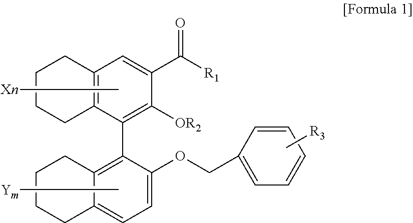
wherein X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1-C10 alkoxy;
Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
n and m are an integer from 0 to 5, respectively;
R1 and R2 are independently C1˜C5 alkyl unsubstituted or substituted with halogen or hydroxyl; C3˜C10 cyclic alkyl unsubstituted or substituted with halogen or hydroxyl; C2-C5 alkenyl unsubstituted or substituted with halogen or hydroxyl; C2˜C5 alkynyl unsubstituted or substituted with halogen or hydroxyl; or aryl unsubstituted or substituted with halogen or hydroxyl;
R3 is —NHCZR4, —NHS(═O)ₐR4, —NHPO(OH)R4 or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1˜C5 perfluoroalkyl, R6 and R7 can form a cycle, and when R3 is —NHC(NH₂)NH₂⁺or —NHCHNH₂⁺, a counter anion is a halogen ion or R10COO⁻ and R10 is C1˜C10 alkyl or C5˜C12 aryl unsubstituted or substituted with C1˜C5 alkyl; and
alkyls described above are linear or branched alkyls.

2. The octahydro-binaphthol derivative according to claim 1, wherein X is independently selected from among hydrogen; halogen; amino; cyano; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
Y is independently selected from among hydrogen; halogen; amino; cyano; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
n and m are an integer from 0 to 5, respectively;
R1 and R2 are independently linear or branched C1˜C5 alkyl unsubstituted or substituted with halogen or hydroxyl; C3˜C10 cyclic alkyl unsubstituted or substituted with halogen or hydroxyl; linear or branched C2˜C5 alkenyl unsubstituted or substituted with halogen or hydroxyl; linear or branched C2˜C5 alkynyl unsubstituted or substituted with halogen or hydroxyl; or aryl unsubstituted or substituted with halogen or hydroxyl; and
R3 is —NHC(═O)NH₂—C₆H₅ or —NHC(NH₂)NH₂⁺.

3. The octahydro-binaphthol derivative according to claim 2, wherein the octahydro-binaphthol derivative of Formula 1 is a compound represented by Formula 2 or 3 below:

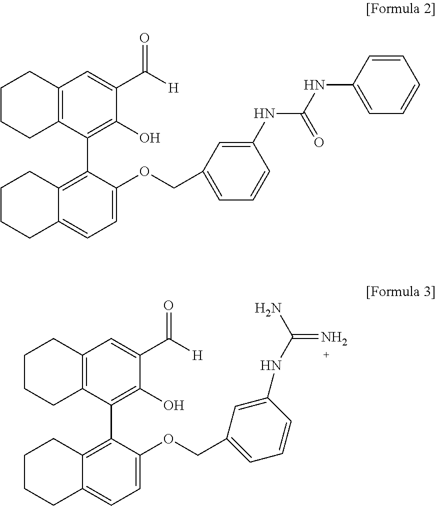

4. The octahydro-binaphthol derivative according to claim 1, wherein the octahydro-binaphthol derivative is provided in an (S) form.

5. The octahydro-binaphthol derivative according to claim 1, wherein the octahydro-binaphthol derivative is provided in an (R) form.

6. The octahydro-binaphthol derivative according to claim 1, wherein the octahydro-binaphthol derivative is used for optical (L/D) conversion of amino acids or optical resolution of amino alcohols.

7. A method of preparing a compound represented by Formula 1 below, comprising reacting a compound represented by Formula 4 below with a compound represented by Formula 5 below in the presence of a base: 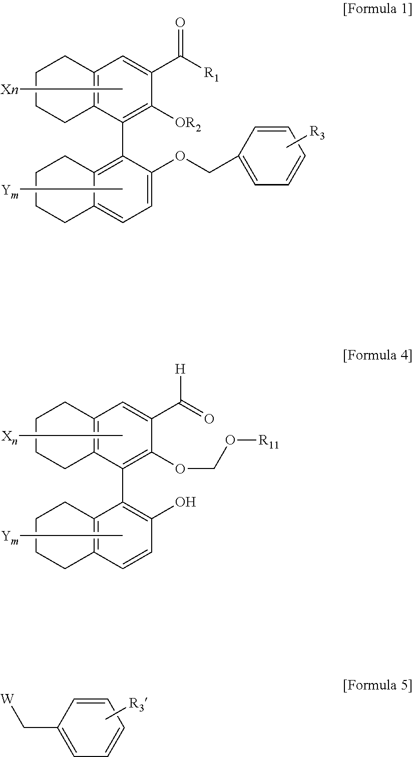
wherein X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
n and m are an integer from 0 to 5, respectively;
R1 and R2 are independently C1˜C5 alkyl unsubstituted or substituted with halogen or hydroxyl; C3˜C10 cyclic alkyl unsubstituted or substituted with halogen or hydroxyl; C2˜C5 alkenyl unsubstituted or substituted with halogen or hydroxyl; C2˜C5 alkynyl unsubstituted or substituted with halogen or hydroxyl; or aryl unsubstituted or substituted with halogen or hydroxyl;
R3 is —NHCZR4, —NHS(═O)ₐR4, —NHPO(OH)R4 or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1˜C5 perfluoroalkyl, R6 and R7 can form a cycle, and when R3 is —NHC(NH₂)NH₂⁺ or —NHCHNH₂⁺, a counter anion is a halogen ion or R10COO⁻ and R10 is C1˜C10 alkyl or C5˜C12 aryl unsubstituted or substituted with C1˜C5 alkyl;
R3′ is independently NO₂, —NHCZR4, —NHS(═O)ₐR4, —NHC(NHBOC)(NBOC) or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1˜C5 perfluoroalkyl, R6 and R7 can form a cycle, and when R3 is —NHC(NH₂)NH₂⁺or —NHCHNH₂⁺, a counter anion is a halogen ion or R10COO⁻ and R10 is C1˜C10 alkyl or C5˜C12 aryl unsubstituted or substituted with C1˜C5 alkyl;
R11 is C1˜C5 alkyl, C6˜C10 arylalkyl or C2˜C10 alkoxyalkyl;
W is halogen; and
alkyls described above are linear or branched alkyls.

8. An intermediate compound represented by Formula 4 below: 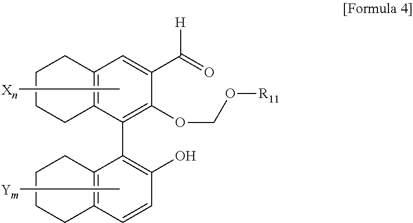
wherein X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
n and m are an integer from 0 to 5, respectively;
R11 is C1˜C5 alkyl, C6˜C10 arylalkyl or C2˜C10 alkoxyalkyl; and
alkyls described above are linear or branched alkyls.

9. A method of preparing a compound represented by Formula 4 below, comprising reacting a compound represented by Formula 6 below with a compound represented by Formula 7 below in the presence of a base: 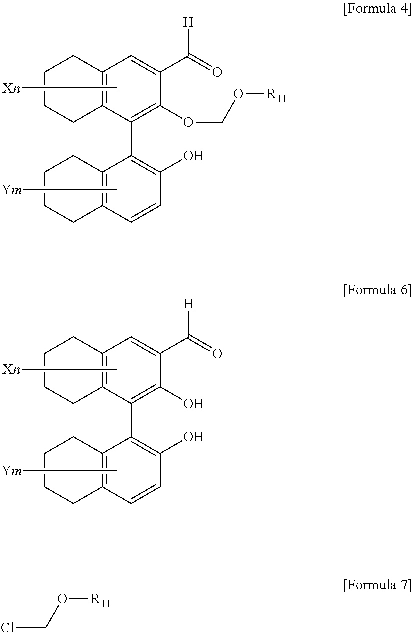
wherein X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C1 alkylcarbonyl; C6˜C10 aryl; and C1˜C10 alkoxy;
n and m are an integer from 0 to 5, respectively;
R11 is C1˜C5 alkyl, C6˜C10 arylalkyl or C2˜C10 alkoxyalkyl; and
alkyls described above are linear or branched alkyls.

10. The method according to claim 9, wherein the compound of Formula 6 is prepared by reacting a compound represented by Formula 8 below with paraformaldehyde.

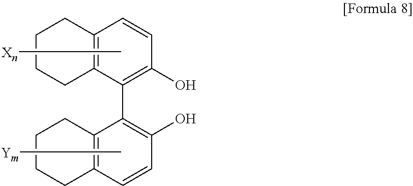

11. A method of optical resolution of racemic amino alcohols or racemic amino acids using a compound represented by Formula 1 below: 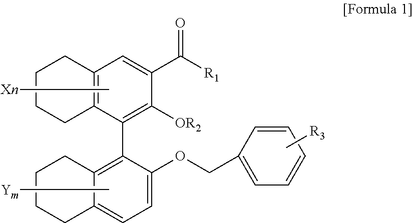
wherein X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
n and m are an integer from 0 to 5, respectively;
R1 and R2 are independently C1˜C5 alkyl unsubstituted or substituted with halogen or hydroxyl; C3˜C10 cyclic alkyl unsubstituted or substituted with halogen or hydroxyl; C2˜C5 alkenyl unsubstituted or substituted with halogen or hydroxyl; C2˜C5 alkynyl unsubstituted or substituted with halogen or hydroxyl; or aryl unsubstituted or substituted with halogen or hydroxyl;
R3 is —NHCZR4, —NHS(═O)ₐR4, —NHPO(OH)R4 or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1-C5 perfluoroalkyl, R6 and R7 can form a cycle, and when R3 is —NHC(NH₂)NH₂⁺ or —NHCHNH₂⁺, a counter anion is a halogen ion or R10COO⁻ and R10 is C1˜C10 alkyl or C5˜C12 aryl unsubstituted or substituted with C1˜C5 alkyl; and
alkyls described above are linear or branched alkyls.

12. A method of optical conversion of amino acids from a D-form to an L-form or vice versa using a compound represented by Formula 1 below: 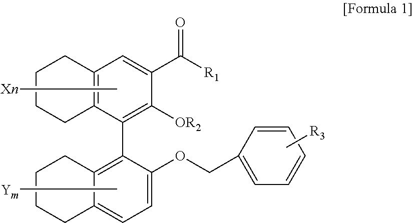
wherein X is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
Y is independently selected from among hydrogen; halogen; amino; nitro; cyano; formyl; carboxyl; C1˜C10 alkyl unsubstituted or substituted with one or more substituents selected from among halogen, hydroxyl, amino, cyano, nitro and C6˜C10 aryl; C1˜C10 alkylcarbonyl; C6˜C10 aryl; and C1˜C10 alkoxy;
n and m are an integer from 0 to 5, respectively;
R1 and R2 are independently C1˜C5 alkyl unsubstituted or substituted with halogen or hydroxyl; C3˜C10 cyclic alkyl unsubstituted or substituted with halogen or hydroxyl; C2˜C5 alkenyl unsubstituted or substituted with halogen or hydroxyl; C2˜C5 alkynyl unsubstituted or substituted with halogen or hydroxyl; or aryl unsubstituted or substituted with halogen or hydroxyl;
R3 is —NHCZR4, —NHS(═O)ₐR4, —NHPO(OH)R4 or —NHC(NHR6)⁺R5, wherein Z is oxygen or sulfur, a is 1 or 2, R4 and R5 are independently hydrogen; C1˜C10 alkyl unsubstituted or substituted with halogen; —NR7R8; or OR9, R6 to R9 are independently hydrogen; C1˜C10 alkyl unsubstituted or substituted with halogen; or C5˜C12 aryl unsubstituted or substituted with one or more substituents selected from among halogen, nitro, C1˜C5 alkyl, C1˜C5 alkoxy and C1-C5 perfluoroalkyl, R6 and R7 can form a cycle, and when R3 is —NHC(NH₂)NH₂⁺ or —NHCHNH₂⁺, a counter anion is a halogen ion or R10COO⁻ and R10 is C1˜C10 alkyl or C5˜C12 aryl unsubstituted or substituted with C1˜C5 alkyl; and
alkyls described above are linear or branched alkyls.
